(19) United States
(12) Patent Application Publication
(10) Pub. No.: US 2021/0069120 A1
(43) Pub. Date: Mar. 11, 2021

(54) COMPOSITE NANOPARTICLE, PREPARATION METHOD THEREOF AND PREPARATION METHOD OF COMPOSITE NANO PREPARATION USING THEREOF

(71) Applicant: JINAN UNIVERSITY, Guangdong (CN)

(72) Inventors: Yu CAI, Guangdong (CN); Qingqing HUANG, Guangdong (CN); Bingyue WANG, Guangdong (CN); Qianwen LI, Guangdong (CN); Ronghua ZHANG, Guangdong (CN); Li YANG, Guangdong (CN); Manling DU, Guangdong (CN); Qianqian MA, Guangdong (CN)

(73) Assignee: JINAN UNIVERSITY, Guangdong (CN)

(21) Appl. No.: 16/481,487

(22) PCT Filed: Aug. 6, 2018
(86) PCT No.: PCT/CN2018/098930

§ 371 Date: Jul. 29, 2019

(30) Foreign Application Priority Data
Aug. 3, 2018 (CN) 201810880186.2

(51) Int. Cl. A61K 9/51; A61K 31/337; A61K 31/37; A61K 36/48

(52) U.S. Cl. CPC A61K 9/5146; A61K 31/337; A61K 2236/15; A61K 36/48; A61K 31/37

(57) ABSTRACT

A composite nanoparticle, a preparation method thereof and preparation method of a composite nano preparation using thereof, wherein the composite nanoparticle is a polymer-lipid nanoparticle encapsulating psoralen, isopsoralen and paclitaxel simultaneously, and the preparation method thereof comprises the following steps of: S1. dissolving soybean lecithin and DSPE-PEG2000 in an aqueous phase, subjected to blending and preheating; and S2. dissolving psoralen, isopsoralen, paclitaxel and PLGA in an oleic phase, subjected to blending and injecting into the aqueous phase of S1 to obtain a mixture, and then heating and blending the mixture to obtain the composite nanoparticle.

### TECHNICAL FIELD

The present invention relates to the field of nano-drugs, and more particularly, to a nano-drug, and a preparation method and an application thereof.

### BACKGROUND

Tumor is one of the major diseases that endanger human health today. Tumor invasion and metastasis is the most common biological behavior and essential feature of a malignant tumor, and is also a key factor affecting the survival and prognosis of tumor patients. Therefore, anti-tumor drugs are the subjects continuously researched and developed by those skilled in the art. In recent years, people have turned to other ways to find an effective method to reverse the multidrug resistance (MDR) of tumor cells. Due to their small volumes and special structures, nanoparticle show unique advantages in improving drug absorption, distribution, metabolism, excretion and toxicity. Therefore, nano drug-delivery systems and the preparation of nanoparticles have received increasing attention. The nano drug-delivery systems comprise a nano liposome, a solid lipid nanoparticle (SLN), a polymer nanoparticle (PLN), a nanosphere, a nanocapsule, a micro emulsion, or the like. The nano drug-delivery systems generally have a particle size between 10 nm and 500 nm, and the MDR of tumors can be reversed by the nano drug-delivery systems. The nano liposome has a biofilm-like structure, and both a lipophilic drug and a hydrophilic drug can be designed into a liposome. The nano liposome has a passive targeting character, and different therapeutic requirements, such as targeting, sustained release, and environmental dependence can be met by modifying the nanoliposome with a polymer. The micro emulsion is an optically isotropic and thermodynamically stable liquid-liquid dispersion system composed of an emulsifier, a co-emulsifier, an oleic phase and an aqueous phase. In the micro emulsion, the drugs have good dispersity. Moreover, the micro emulsion has a solubilization effect on indissolvable drugs, thus improving the bioavailability. The SLN is a nano preparation prepared with lipid materials, and the SLN can promote the drugs to penetrate a blood-brain barrier enriched with P-gp. Many researches are basically focused on the preparation of chemotherapy drugs SLN and the study of the action mechanisms thereof. In the prior art, anti-P-gp micromolecular inhibitors (such as verapamil, cyclosporine A, GG918, etc.) and the anti-tumor drugs are jointly used to prepare the SLN. The PLN is a hydrophilic micelle prepared by polymerization with sodium bis(2-ethylhexyl) sulfosuccinate as a monomer. Since non-degradable materials have a potential biological toxicity, a biodegradable PLN capable of being oriented to a lysosome is gradually prepared with poly(methyl cyanoacrylate), polystyrene, polyamide and other materials.

Researches and products of chemotherapy drug nanoparticles have been reported and listed. For example, Abraxane is the first non-dissolved nano-albumin bound chemotherapy drug, which is a nanoparticle of paclitaxel coated with albumin for treating breast cancer metastasis. Gliadelwafer is a nanoparticle of carmustine coated with polifeprosan 20 for treating highly-differentiated malignant neurospongioma. DaunoXome is a daunorubicin liposome for treating Kaposi's sarcoma. Myocet is a doxorubicin liposome combined with cyclophosphamide for treating metastatic breast cancer. DOXIL is a doxorubicin liposome for treating metastatic ovarian cancer. A copolymer PK2 of adriamycin galactosamine and N-(2-hydroxypropyl)isobutylamide for treating liver cancer. The various pharmaceutical preparations of the drug-loading systems above have certain effects of reversing MDR activity, but also have some problems, such as low drug loading amount, poor stability and easy leakage, etc.

The application of the biodegradable PLN can effectively improve the drug stability, thus playing the roles of sustained release and controlled release. Currently available biodegradable carrier materials mainly comprise biodegradable high-molecular polymers and natural macromolecular systems. The biodegradable high-molecular polymers comprise polylactide (PLA), polyglycolide (PLG), polylactide-glycolide (PLGA), polycaprolactone (PCL), polyorthoester (POE), polyalkylcyanoacrylate (PACA), polyvinylpyrrolidone (PVP), etc. The natural macromolecular systems comprise protein, polysaccharide, gelatin, polyacrylic starch, chitin and derivatives, sodium alginate, gelatin, albumin, lecithin, cholesterol, etc. As for the polymers above, the polylactide-glycolide (PLGA) is a high-molecular compound polymerized by lactic acid and glycolic acid, and has the advantages of low toxicity, good granulation and biocompatibility. The PLGA carrier materials can be degraded in a water-soluble system through the cleavage of ester bonds, the lactic acid and the glycolic acid produced by degradation are further degraded into water and carbon dioxide in vivo and finally discharged in vitro, and the release of an encapsulated drug is regulated through a degradation rate of the PLGA itself, so that the drug can be released at a stable rate for a long time, and a stable blood concentration can be maintained. The PLGA can increase a water solubility of a fat-soluble drug and improve a bioavailability of the fat-soluble drug. The PLGA has been approved by FDA to be used in drug carriers and other fields. The degradation rate of the PLGA can be affected and changed by changing the ratio of the lactic acid and the glycolic acid in the PLGA. At present, the more mature preparation method of the PLGA nanoparticles is a double emulsion solvent volatilization method. Only the preparation method and physicochemical properties of the chemotherapy drugs have been reported in the study of the PLGA nanoparticle preparation, while the researches on a drug-resistance reversal agent encapsulated with the PLGA nano-carrier and the application thereof to resisting the MDR of tumors have not been reported yet.

As an effective component with strong liposolubility extracted from leguminosae plants, psoralen (PSO) is a calcium channel blocker, and can inhibit the pumpout of P-gp proteins and assist the chemotherapy drugs to reverse the MDR effects of the tumor cells at the same time. Paclitaxel is a diterpene alkaloid compound with an anti-cancer activity, which is widely used in clinical treatment of breast cancer, ovarian cancer and lung cancer. At present, both the psoralen and the paclitaxel have separate effects on the treatment of breast cancer, but the psoralen has a poor water solubility and a low bioavailability, while the paclitaxel has side effects such as bone marrow suppression, allergic reaction, cardiac toxicity and pneumonia. Therefore, it is necessary to find an effective preparation to increase the solubility of the psoralen, improve the bioavailability, reduce the toxic and side effects of the paclitaxel, and enhance the anti-tumor effect. Meanwhile, for different active drugs, there are significant differences in the encapsulation efficiency and the stability of the PLN preparation. For specific PLN preparations, how to improve the encapsulation efficiency and the stability of the active drugs is still the focus, the key point and the difficulty of the current research.

### SUMMARY

The technical problem to be solved by the present invention is to overcome the defects and deficiencies of the anti-tumor drugs and the PLN preparations in the prior art, and provide a composite nanoparticle with high encapsulation efficiency and stability, which can effectively improve the drug effect and reverse the MDR of tumors.

A first object of the present invention is to provide a composite nanoparticle.

A second object of the present invention is to provide a preparation method of the composite nanoparticle.

A third object of the present invention is to provide an application of the composite nanoparticle.

The above-mentioned objects of the present invention are achieved by the following technical solutions.

A composite nanoparticle is a polymer-lipid nanoparticle encapsulating psoralen, isopsoralen and paclitaxel simultaneously.

Preferably, the composite nanoparticle has a particle size of 96.89±2.12 nm.

A preparation method of the above-mentioned composite nanoparticle comprises the following steps of:

S1. dissolving soybean lecithin and DSPE-PEG2000 in an aqueous phase, subjected to blending and preheating; and

S2. dissolving psoralen, isopsoralen, paclitaxel and PLGA in an oleic phase, subjected to blending and injecting into the aqueous phase of the S1, and then heating and blending a mixture to obtain the composite nanoparticle.

The composite nanoparticle according to the present invention is a novel nano drug-delivery system based on lipid nanoparticles and polymer nanoparticles. Structurally, the lipid polymer nanoparticle can be divided into hydrophobic polymer cores and hydrophilic shells formed by lipid molecular layers, which improve the stability of nanoparticles while enhancing the cell uptake and improving the encapsulation efficiency. A polymer, polylactic acid-glycolic acid copolymer (PLGA), for preparing the lipid polymer nanoparticles has good biocompatibility; an amphiphilic phospholipid has a hydrophilic group and a hydrophobic chain. In a self-assembled process, the hydrophobic chain of the phospholipid forms a hydrophobic core, in which polymers or drugs are encapsulated. The hydrophilic group forms a lipid monomolecular layer or bimolecular layer. Alternatively, a polyethylene glycol-lipid (DSPE-PEG₂₀₀₀) can be added and embedded into a lipid monolayer to form a polyethylene glycol invisible layer outside the lipid shell, thus improving the electrostatic and spatial stabilities and prolonging the cycle time.

Preferably, the aqueous phase is absolute ethyl alcohol.

Preferably, the oleic phase is acetonitrile.

Preferably, a mass ratio of the soybean lecithin to the DSPE-PEG2000 in the S1 is 5-6:1.

Preferably, a mass ratio of the psoralen to the isopsoralen to the paclitaxel in the S2 is 2:2:1.

Preferably, a mass ratio of the psoralen to the isopsoralen to the paclitaxel to PLGA is 2-4:2-4:1-2:10.

Preferably, the preheating in the Si refers to heating at 70° C. for 3 minutes.

Preferably, the heating and blending in the S2 refers to heating with stirring at 70° C. for 90 minutes.

The present invention also requests to protect an application of the composite nanoparticle in preparing anti-tumor drugs.

Preferably, the tumor is breast cancer.

Compared with the prior art, the present invention has the following advantageous effects.

The present invention provides a polymer-lipid nanoparticle encapsulating psoralen, isopsoralen and paclitaxel simultaneously. The nanoparticle has high structure integrity, good stability and storability as well as high stability and biocompatibility, has an empty sustained-release effect and an encapsulation efficiency of more than 80%, can effectively improve the drug effect, resist breast cancer tumor metastasis, and has a wider application prospect in cancer treatment.

### BRIEF DESCRIPTION OF THE DRAWINGS

FIG. 1 shows a growth inhibition effect of PTX on a MDA-MB-231 (48 hours).

FIG. 2 shows a proliferation inhibition effect of PSO on a MDA-MB-231 cell.

FIG. 3 shows a proliferation inhibition effect of I-PSO on a MDA-MB-231 cell.

FIG. 4 shows a toxic effect of Blank PLN on a MDA-MB-231 cell (48 hours).

FIG. 5 shows influences of PTX, PTX+PSO+I-PSO and (PTX+PSO+I-PSO)-PLN on a proliferation inhibition effect on a MDA-MB-231 cell.

FIG. 6 shows results of apoptosis detection after treating MDA-MB-231 cells with different drug components, wherein the first row refers to blank control, Blank PLN, PTX and PSO in sequence from left to right, while the second row refers to I-PSO, PTX+PSO+I-PSO and (PTX+PSO+I-PSO)-PLN in sequence from left to right.

FIG. 7 shows results of scratch detection of MDA-MB-231 cells under different drug components.

FIG. 8 shows results of invasion detection of MDA-MB-231 cells under different drug components.

### DETAILED DESCRIPTION

The invention is further described hereinafter with reference to the accompanying drawings and specific embodiments, but the embodiments are not intended to limit the invention in any form. Unless otherwise indicated, the reagents, methods, and devices employed in the invention are routine reagents, methods, and devices in the art.

The reagents and materials used in the following embodiments are commercially available unless otherwise stated.

Cell culture of the present invention is carried out according to the following steps.

1. Proliferation and Passage of Cells

A human breast cancer sensitive strain MDA-MB-231 and a drug resistant cell strain thereof used in the invention are cultured in a constant temperature (37° C.) CO₂ incubator with a CO₂ concentration of 5% and a humidity of 95%, and a medium used is a conventional DMEM medium containing 10% fetal bovine serum and 1% streptomycin double antibody. A color change of the medium in a culture flask is observed under a microscope every day to check whether there is turbidity, observing a cell state including cell morphology, density and spreading, and a liquid is changed after the color of the medium becomes light. If cell contamination is found during the cell culture, all the cells are discarded immediately, and the cells are thoroughly disinfected, and then resuscitated. The cells cannot be used for experiment unless the cells are in good condition.

(1) Cells are observed under the microscope every day until the cells grow to a saturation of 80% to 90%, and prepared for cell passage.

(2) Ultraviolet radiation disinfection is carried out on a super clean bench and articles used in the experiment for 30 minutes.

(3) A culture fluid is removed, and PBS (2 mL/flask) is added, then the culture fluid and cells remaining in the culture flask are washed carefully.

(4) The PBS is removed, and 1 mL of 0.25% trypsin containing EDTA is added to each flask, and stood in an incubator for digestion for 2 to 3 minutes.

(5) Cell changes are observed under the microscope until the cells shrink and become round, the cell gap increases, and some cells fall off, then it can be deemed that the cells are completely digested. Cell medium containing serum and in an amount equal to the pancreatin is added to terminate the digestion, and the cells are repeatedly blown and beaten by a Pasteur pipette until the cells adhered to a flask wall fall off completely.

(6) A mixture of the blown and beaten cells and the pancreatin medium is transferred to a 15 mL centrifuge tube and centrifuged at L000 rpm for 5 minutes.

(7) A supernatant in the centrifuge tube is discarded, and white precipitates at the bottom of the centrifuge tube need to be protected as centrifuged cells and prevented from discarding, 2 mL of fresh cell culture fluid is added into the centrifuge tube, and repeatedly blown and beaten by a Pasteur pipette for 40 times to resuspend the cells to form a single cell suspension.

(8) 1 mL of the cells resuspended into the single cell suspension are sucked up and added to a new 25 cm² cell culture flask, and then 3 mL of fresh medium is added to the culture flask.

(9) 1 mL of the resuspended cells is respectively sucked up and, added to the prepared culture flask mentioned above, and then the culture flask is gently shaken from left to right as well as up and down after leveling, so that the cells are evenly covered on the bottom surface of the culture flask.

(10) The cells are observed under the microscope, cell names and passage time are marked, and then the cells are placed in an incubator again for continuous culture; after the super clean bench is tidied up, a top of the super clean bench is wiped with 75% alcohol, then an alcohol lamp is turned off, and ultraviolet disinfection is performed.

2. Counting of Cells

(1) A cell counting plate and a coverslip are wiped clean with 75% alcohol. After shaking left and right on an alcohol lamp flame for several times and drying, the coverslip is completely covered on the counting plate and kept flat, so that the coverslip cannot slide.

(2) Trypsin-digested cells are added according to a cell passage method, the cells are collected and transferred into a 10 mL sterile centrifuge tube and preparation of a single cell suspension is performed.

(3) 10 μL of the single cell suspension is sucked up by a pipette and then carefully injected between the counting plate and the coverslip.

(4) Cells in the four quadrants of the cell counting plate are counted under a microscope.

(5) Cell counting results are recorded and a cell density of each flask is calculated.

3. Cryopreservation of Cells

(1) 3 to 4 generations of cells with good growth state are selected for cryopreservation, and the medium of the cells is replaced 24 hours before the cryopreservation of cells.

(2) The medium in the culture flask is sucked out, the cells in the flask are washed with PBS, the culture flask is flattened, gently shaken up and down as well as left and right, and then the cells are washed carefully to reduce medium residuals.

(3) Conventional digestion is performed on the cells, a DMEM high-sugar medium containing serum is added when the cells are completely digested to terminate the digestion process, and then the cells are repeatedly blown and beaten into a single cell suspension by a Pasteur pipette.

(4) The blown and beaten cell suspension is transferred to a 15 mL centrifuge tube with a pipette and centrifuged at 1500 rpm for 5 minutes.

(5) A supernatant is removed with a pipette, and the cells at the bottom of the centrifuge tube need to be protected, then a newly prepared cell cryopreservation fluid is added, and the mixture is blown and beaten by a Pasteur pipette to form a single cell suspension.

(6) 1 mL of the blown and beaten cell suspension is sucked up with a pipette, and sub-packaged into a cell cryopreservation tube, wherein 1 mL of cell suspension is sub-packaged for each tube, and then the tube is sterilized with an alcohol lamp, and sealed with a sealing film.

(7) Cryopreservation information is recorded on a wall of the cryopreservation tube, including cell name, number of generations and date of cryopreservation.

(8) The cryopreservation tubes with sub-packaged cells are placed in a refrigerator at 4° C. for 30 minutes and then put in a refrigerator at 20° C. below zero for 1 to 2 hours, then the cryopreserved cells are put in a cryopreservation box, recorded, transferred to a refrigerator at 80° C. below zero for cryopreservation, and then transferred to a liquid nitrogen tank for storage the next day.

### Embodiment 1 Preparation of a Polymer-Lipid Nanoparticle (PLN)

1. Preparation of a Reaction Fluid

Soybean lecithin (PC): a proper amount of PC was weighed and dissolved in absolute ethyl alcohol to prepare a solution with a concentration of 40 mg/ml;

DSPE-PEG2000: a proper amount of DSPE was weighed and dissolved in absolute ethyl alcohol to prepare a solution with a concentration of 5 mg/ml;

psoralen (PSO): a proper amount of psoralen was weighed and dissolved in acetonitrile to prepare a solution with a concentration of 6 mg/ml;

isopsoralen (I-PSO): a proper amount of psoralen was weighed and dissolved in acetonitrile to prepare a solution with a concentration of 6 mg/ml;

paclitaxel (PTX): a proper amount of psoralen was weighed and dissolved in acetonitrile to prepare a solution with a concentration of 6 mg/ml; and

PLGA: a proper amount of PLGA was weighed and dissolved in acetonitrile to prepare a solution with a concentration of 10 mg/ml.

2. Preparation Method

S1. 18 mL of absolute ethyl alcohol was weighed by a measuring cylinder and added to a beaker, 25.5 mg of soybean lecithin (PC) and 4.5 mg of DSPE-PEG2000 were added according to a corresponding volume calculated on the basis of the concentration of the solution prepared in S1, blended, and preheated at 70° C. for 3 minutes.

S2. 2 mg of psoralen, 2 mg of isopsoralen and 1 mg of paclitaxel (a mass ratio of 2:2:1) were blended with 10 mg of PLGA according to the corresponding volume calculated on the basis of the concentration of the solution prepared in S1, then injected into an aqueous phase of S1 with an entry needle, heated and stirred at 70° C. for 90 minutes to prepare and obtain polymer-lipid nanoparticles, (PTX+PSO+I-PSO)-PLN, encapsulating the three drugs including psoralen, isopsoralen and paclitaxel simultaneously.

Meanwhile, blank polymer-lipid nanoparticles, Blank PLN, not encapsulating drugs were prepared and obtained according to the above preparation method, but the only difference was that, in S2, only PLGA was added into the aqueous phase of S1.

2. Results

The prepared nanoparticles were about 96 nm with a PDI of 0.244, indicating that the nanoparticles have good particle size dispersity. A Zeta potential was about −25 mV, indicating that the electrostatic repulsion between the nanoparticles was large, which was conducive to maintaining the stability of a solution system and preventing nanoparticles from aggregation and precipitation. When placing the PLNs in a PBS, a RPMI1640 medium, a complete medium of 10% fetal bovine serum and 10% (v/v) human plasma, incubating at 37° C. for 120 hours, the particle size and polydispersity of the nanoparticles did not change significantly, indicating that a PEGylated lipid monomolecular layer stabilized a polymer core and prevented the nanoparticles from aggregating within 120 hours, and the stability was high. Upon tests, encapsulation efficiency of the drug was as high as 82%. The composite nanoparticles prepared above were subjected to the cell tests described in Embodiments 4 to 7.

### Embodiment 2

1. Preparation of a Reaction Fluid

Soybean lecithin (PC): a proper amount of PC was weighed and dissolved in absolute ethyl alcohol to a solution with a concentration of 40 mg/ml;

DSPE-PEG2000: a proper amount of DSPE was weighed and dissolved in absolute ethyl alcohol to a solution with a concentration of 5 mg/ml;

psoralen (PSO): a proper amount of psoralen was weighed and dissolved in acetonitrile to prepare a solution with a concentration of 6 mg/ml; and

isopsoralen (I-PSO): a proper amount of psoralen was weighed and dissolved in acetonitrile to prepare a solution with a concentration of 6 mg/ml; and

paclitaxel (PTX): a proper amount of psoralen was weighed and dissolved in acetonitrile to prepare a solution with a concentration of 6 mg/ml; and

PLGA: a proper amount of PLGA was weighed and dissolved in acetonitrile to prepare a solution with a concentration of 10 mg/ml.

2. Preparation Method

S1. 18 mL of absolute ethyl alcohol was weighed by a measuring cylinder and added to a beaker, 27 mg of soybean lecithin (PC) and 4.5 mg of DSPE-PEG2000 were added according to a corresponding volume calculated on the basis of the concentration of the solution prepared in S1, blended, and preheated at 70° C. for 3 minutes.

S2. 2 mg of psoralen, 2 mg of isopsoralen and 1 mg of paclitaxel (a mass ratio of 2:2:1) were blended with 10 mg of PLGA according to the corresponding volume calculated on the basis of the concentration of the solution prepared in S1, then injected into an aqueous phase of S1 with an entry needle, heated and stirred at 70° C. for 90 minutes to prepare and obtain polymer-lipid nanoparticles, (PTX+PSO+I-PSO)-PLN, encapsulating the three drugs including psoralen, isopsoralen and paclitaxel simultaneously.

2. Results

The prepared nanoparticles were about 95 nm with a PDI of 0.249, indicating that the nanoparticles have good particle size dispersity. A Zeta potential was about −26 mV, indicating that the electrostatic repulsion between the nanoparticles was large, which was conducive to maintaining the stability of a solution system and preventing nanoparticles from aggregation and precipitation. When placing the PLNs in a PBS, a RPMI1640 medium, a complete medium of 10% fetal bovine serum and 10% (v/v) human plasma, incubating at 37° C. for 120 hours, the particle size and polydispersity of the nanoparticles did not change significantly, indicating that a PEGylated lipid monomolecular layer stabilized a polymer core and prevented the nanoparticles from aggregating within 120 hours, and the stability was high. Upon tests, encapsulation efficiency of the drug was as high as 85%.

### Embodiment 3

1. Preparation of Reaction Fluid

Soybean lecithin (PC): a proper amount of PC was weighed and dissolved in absolute ethyl alcohol to a solution with a concentration of 40 mg/ml;

DSPE-PEG2000: a proper amount of DSPE was weighed and dissolved in absolute ethyl alcohol to a solution with a concentration of 5 mg/ml;

psoralen (PSO): a proper amount of psoralen was weighed and dissolved in acetonitrile to prepare a solution with a concentration of 6 mg/ml; and

isopsoralen (I-PSO): a proper amount of psoralen was weighed and dissolved in acetonitrile to prepare a solution with a concentration of 6 mg/ml; and

paclitaxel (PTX): a proper amount of psoralen was weighed and dissolved in acetonitrile to prepare a solution with a concentration of 6 mg/ml; and

PLGA: a proper amount of PLGA was weighed and dissolved in acetonitrile to prepare a solution with a concentration of 10 mg/ml.

2. Preparation Method

S1. 18 mL of absolute ethyl alcohol was weighed by a measuring cylinder and added to a beaker, 25.5 mg of soybean lecithin (PC) and 4.5 mg of DSPE-PEG2000 were added according to a corresponding volume calculated on the basis of the concentration of the solution prepared in S1, blended, and preheated at 70° C. for 3 minutes.

S2. 4 mg of psoralen, 4 mg of isopsoralen and 2 mg of paclitaxel (a mass ratio of 2:2:1) were blended with 10 mg of PLGA according to the corresponding volume calculated on the basis of the concentration of the solution prepared in S1, then injected into an aqueous phase of S1 with an entry needle, heated and stirred at 70° C. for 90 minutes to prepare and obtain polymer-lipid nanoparticles, (PTX+PSO+I-PSO)-PLN, encapsulating the three drugs including psoralen, isopsoralen and paclitaxel simultaneously.

2. Results

The prepared nanoparticles were about 102 nm with a PDI of 0.25, indicating that the nanoparticles have good particle size dispersity. A Zeta potential was about −27 mV, indicating that the electrostatic repulsion between the nanoparticles was large, which was conducive to maintaining the stability of a solution system and preventing nanoparticles from aggregation and precipitation. When placing the PLNs in a PBS, a RPMI1640 medium, a complete medium of 10% fetal bovine serum and 10% (v/v) human plasma, incubating at 37° C. for 120 hours, the particle size and polydispersity of the nanoparticles did not change significantly, indicating that a PEGylated lipid monomolecular layer stabilized a polymer core and prevented the nanoparticles from aggregating within 120 hours, and the stability was high. Upon tests, encapsulation efficiency of the drug was as high as 87%.

### Embodiment 4 Effects of Different Drugs or Preparations on MDA-MB-231

1. Proliferation Inhibition Effect of Paclitaxel (PTX) on MDA-MB-231

4×10³ MDA-MB-231 cells/well were inoculated into a 96-well plate with a concentration of 100 μL/well, and cultured in a 5% CO₂ cell incubator at 37° C. for 24 hours, then a supernatant was removed after 24 hours, and 200 μL of drug-containing medium was added. The final concentrations of the PTX added to the MDA-MB-231 cells were 0.0039 μmol/L, 0.0078 μmol/L, 0.0156 μmol/L, 0.03125 μmol/L, 0.0625 μmol/L, 0.125 μmol/L, 0.25 μmol/L, 0.5 μmol/L and 1 μmol/L. At the same time, a control group (the same amount of culture fluid was added) and a blank zeroing group were set up. Each well was provided with 6 multiple wells and placed in a CO₂ incubator for continuous cultivation for 48 hours. MTT solution in a concentration of 20 μL/well (5 mg/mL) was added 4 hours before the experiment was terminated, then 150 μL of DMSO was added to each well after 4 hours, and sufficiently oscillated for 10 minutes. An OD value was measured at 570 nm of an enzyme reader.

Zero adjustment was performed based on the blank group and the experiment was repeated for three times to calculate a cell viability and IC50:

Cell viability=(OD value of drug treatment group/OD value of control group)×100%

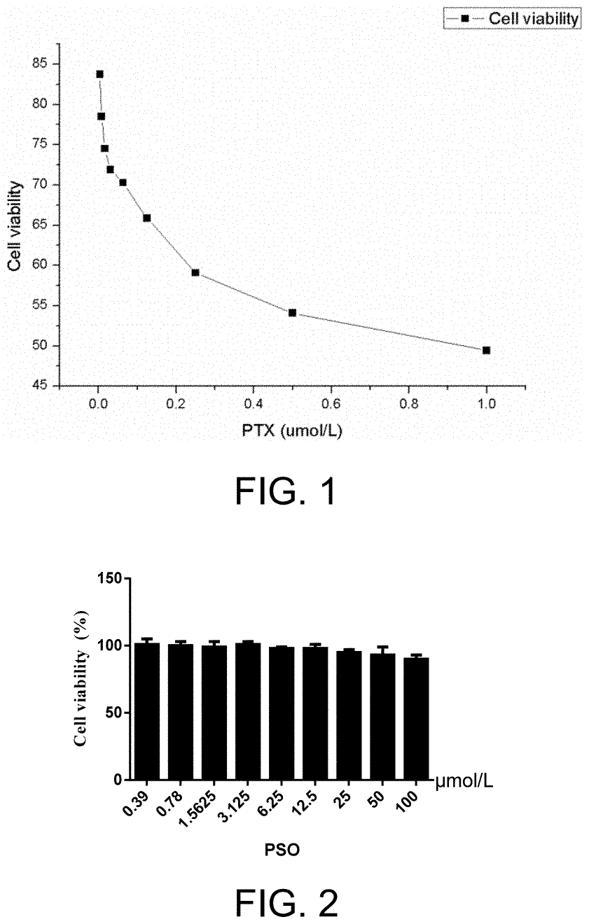

The results are as shown in FIG. 1, wherein the cell viability decreases with the increase of the PTX concentration, and the IC50 is 1.02 μmol/l.

2. Proliferation Inhibition Effect of Psoralen (PSO) on MDA-MB-231

4×10³ MDA-MB-231 cells/well were inoculated into a 96-well plate with a concentration of 100 μL/well, and cultured in a 5% CO₂ cell incubator at 37° C. for 24 hours, then a supernatant was removed after 24 hours, and 200 μL of drug-containing medium was added. The final concentrations of the PSO added to the MDA-MB-231 cells were 100 μmol/L, 50 μmol/L, 25 μmol/L, 12.5 μmol/L, 6.25 μmol/L, 3.125 μmol/L, 1.5625 μmol/L, 0.78 μmol/L and 0.39 μmol/L. At the same time, a control group (the same amount of culture fluid was added) and a blank zeroing group were set up. Each well was provided with 6 multiple wells and placed in a CO₂ incubator for continuous cultivation for 48 hours. MTT solution in a concentration of 20 μL/well (5 mg/mL) was added 4 hours before the experiment was terminated, then 150 μL of DMSO was added to each well after 4 hours, and sufficiently oscillated for 10 minutes. An OD value was measured at 570 nm of an enzyme reader.

Zero adjustment was performed based on the blank group and the experiment was repeated for three times to calculate a cell viability:

Cell viability=(OD value of drug treatment group/OD value of control group)×100%

The results are as shown in FIG. 2, wherein the PSO has no obvious toxicity to the MDA-MB-231 cells for 48 hours in a concentration range of 0.39 to 100 μmol/L.

3. Proliferation Inhibition Effect of Isopsoralen (IPSO) on MDA-MB-231

4×10³ MDA-MB-231 cells/well were inoculated into a 96-well plate with a concentration of 100 μL/well, and cultured in a 5% CO₂ cell incubator at 37° C. for 24 hours, then a supernatant was removed after 24 hours, and 200 μL of drug-containing medium was added. The final concentrations of the IPSO added to the MDA-MB-231 cells were 100 μmol/L, 50 μmol/L, 25 μmol/L, 12.5 μmol/L, 6.25 μmol/L, 3.125 μmol/L, 1.5625 μmol/L, 0.78 μmol/L and 0.39 μmol/L. At the same time, a control group (the same amount of culture fluid was added) and a blank zeroing group were set up. Each well was provided with 6 multiple wells and placed in a CO₂ incubator for continuous cultivation for 48 hours. MTT solution in a concentration of 20 μL/well (5 mg/mL) was added 4 hours before the experiment was terminated, then 150 μL of DMSO was added to each well after 4 hours, and sufficiently oscillated for 10 minutes. An OD value was measured at 570 nm of an enzyme reader.

Zero adjustment was performed based on the blank group and the experiment was repeated for three times to calculate a cell viability:

Cell viability=(OD value of drug treatment group/OD value of control group)×100%

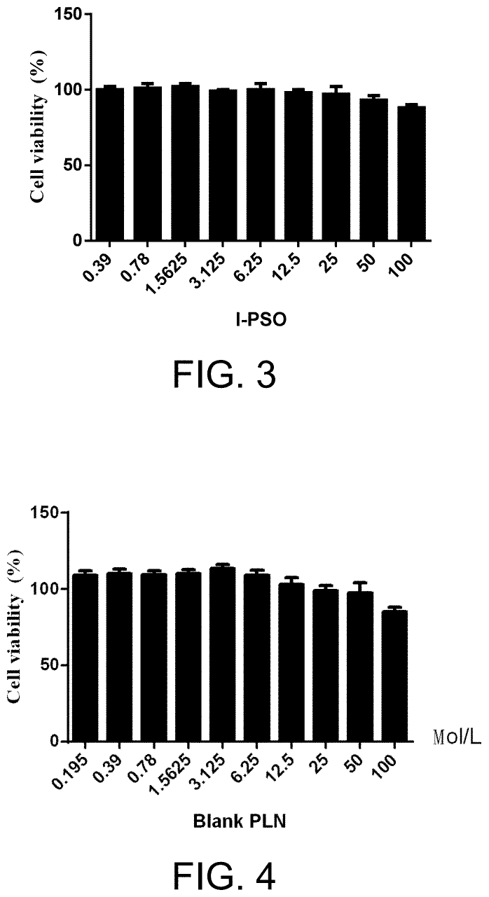

The results are as shown in FIG. 3, wherein the PSO has no obvious toxicity to the MDA-MB-231 cells for 48 hours in a concentration range of 0.39 to 100 μmol/L.

4. Toxic Effect of Blank PLN on a MDA-MB-231 Cell

4×10³ MDA-MB-231 cells/well were inoculated into a 96-well plate with a concentration of 100 μL/well, and cultured in a 5% CO₂ cell incubator at 37° C. for 24 hours, then a supernatant was removed after 24 hours, and 200 μL of drug-containing medium was added. Based on psoralen, the final concentrations of the blank PLN added to the MDA-MB-231 cells were 200 μmol/L, 100 μmol/L, 50 μmol/L, 25 μmol/L, 12.5 μmol/L, 6.25 μmol/L, 3.125 μmol/L, 1.5625 μmol/L and 0.78 μmol/L. At the same time, a control group (the same amount of culture fluid was added) and a blank zeroing group were set up. Each well was provided with 6 multiple wells and placed in a CO₂ incubator for continuous cultivation for 48 hours. MTT solution in a concentration of 20 μL/well (5 mg/mL) was added 4 hours before the experiment was terminated, then 150 μL of DMSO was added to each well after 4 hours, and sufficiently oscillated for 10 minutes. An OD value was measured at 570 nm of an enzyme reader.

Zero adjustment was performed based on the blank group and the experiment was repeated for three times to calculate a cell viability:

Cell viability=(OD value of drug treatment group/OD value of control group)×100%

The results are as shown in FIG. 4, wherein the blank PLN has no obvious toxicity to the MDA-MB-231 sensitive cell lines in a concentration range of 0.39 to 100 mol/l.

5. Influences of PTX, PTX+PSO+I-PSO and (PTX+PSO+I-PSO)-PLN on a Proliferation Inhibition Effect on a MDA-MB-231 Cell

Different doses of PTX, PTX+PSO+I-PSO and (PTX+PSO+I-PSO)-PLN were added to the cells respectively, and placed in a CO₂ incubator for continuous culture for 48 hours. OD values were detected by an enzyme reader and cell viabilities were calculated.

Cell viability=(OD value of drug treatment group/OD value of control group)×100%

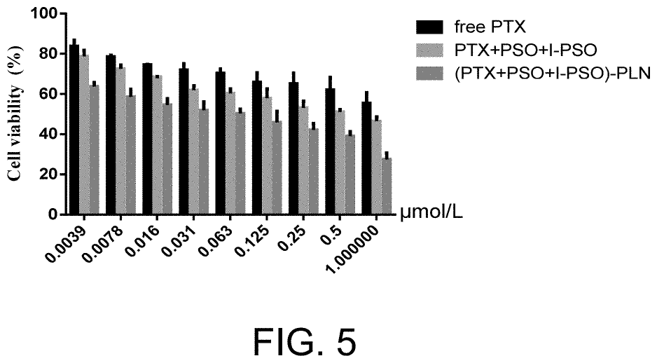

The results are as shown in FIG. 5. Compared with the PTX and PTX+PSO+I-PSO groups, the IC50 of the (PTX+PSO+I-PSO)-PLN on the MDA-MB-231 cells is 0.063 μmol/L, indicating that the toxicity thereof to cells is significantly enhanced.

### Embodiment 5 Apoptosis Effect of Different Drug Components on MDA-MB-231 Cells Detected by a Flow Cytometry

1. Method

After Blank PLN, PTX, PSO, I-PSO, PTX+PSO+I-PSO and (PTX+PSO+I-PSO)-PLN were used to act on the MDA-MB-231 cells respectively, a blank control without drug treatment was set at the same time. Cells were collected, an Annexin V-FITC buffer solution and a propidium iodide (PI) staining solution were added respectively, and cell apoptosis was detected by a flow cytometry, wherein green fluorescence was Annexin V-FITC, and red fluorescence was PI. Annexin V-FITC/PI double staining was dominant in late apoptotic cells and dead cells which referred to an upper right region on a flow cytometry diagram, while AnnexinV-FITC staining was dominant in early apoptotic cells which referred to a lower right region on the flow cytometry diagram. The experiment was repeated for three times.

2. Results

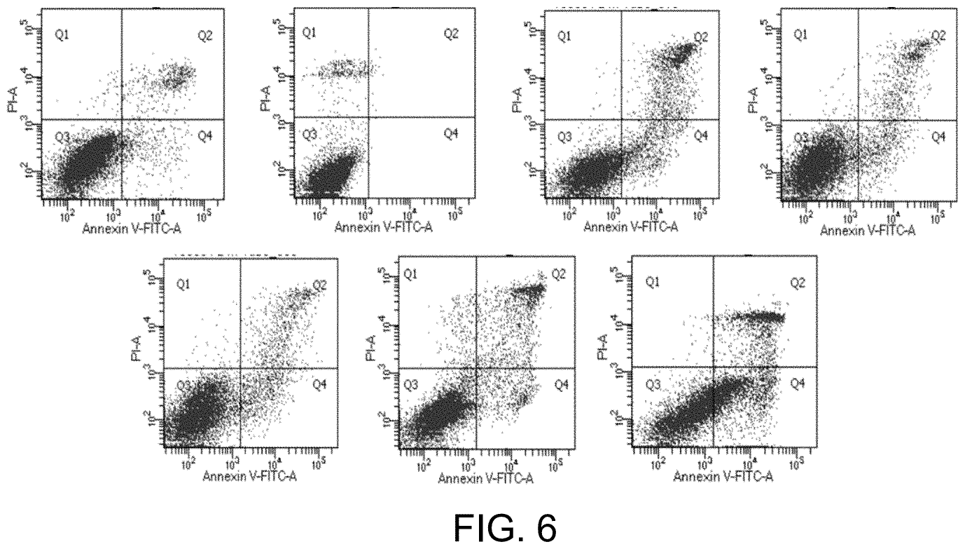

The detection results are as shown in FIG. 6 and Table 1. The apoptotic rate of the blank PLN is 0.3%, indicating that blank PLN has no obvious toxic effect on the cells. This is consistent with the results of the MTT experiment. In addition to the blank PLN, compared with the blank control group, different administration groups have the effect of promoting the apoptosis of the MDA-MB-231 cells, especially the (PTX+PSO+I-PSO)-PLN group which has an apoptosis rate of 45.3%.

TABLE 1
Apoptosis rate (%) of MDA-MB-231 cells after
being treated with different drug components
Apoptosis rate/group

| Blank | Blank | | | | PTX + PSO + | (PTX + PSO + |
|---|---|---|---|---|---|---|
| control | PLN | PTX | PSO | I-PSO | I-PSO | I-PSO) − PLN |
| 9.9 | 0.3 | 15.1 | 13.8 | 11.5 | 25.6 | 45.3 |

### Embodiment 6 Scratch Detection of MDA-MB-231 Cells Under Different Drug Components

Human breast cancer cells MDA-MB-231 were digested with 0.25% EDTA trypsin, and then the cells were blown down with a fresh culture fluid to prepare a cell suspension. The cells were counted by using a blood counting plate and a cell concentration was adjusted to 2×10⁵/mL. The cells were laid in a 12-well plate overnight according to a concentration of 2×10⁵cells/well. A scratch line was drawn on a bottom of the 12-well plate in advance. When the cells grew to 90% confluence, the cells were scratched by a sterilized 20 μL pipettor nozzle, and each well was scratched once on a middle line. The plate was washed for three times with PBS to remove the scraped cells. The 12 wells on the plate were divided into four groups with three repetitions in each group. Serum-free culture fluids containing the following drug components of PTX, PSO, I-PSO, PTX+PSO+I-PSO and (PTX+PSO+I-PSO)-PLN were respectively added to induce, and a cell culture plate was placed under a 10×10 microscope to photograph, and the scratch line was taken as a starting point during photographing to take a width of a scratch as a width at 0 hour.

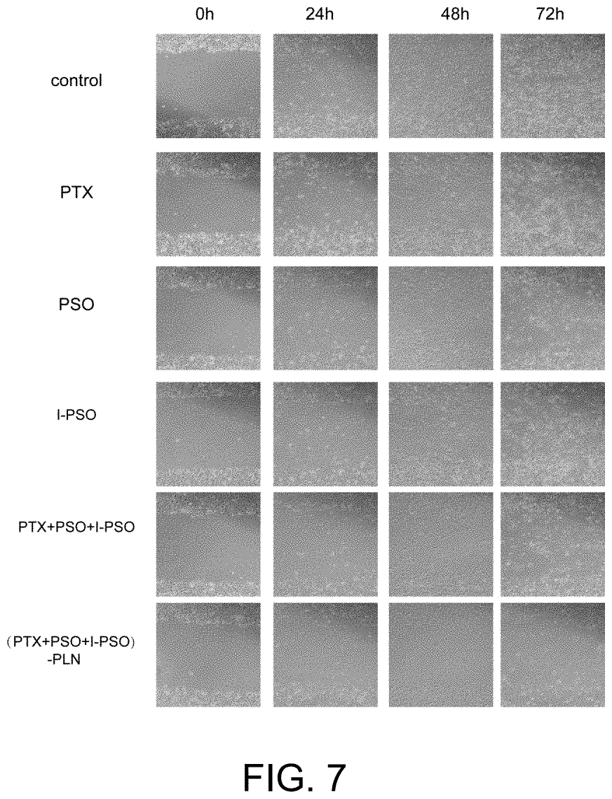

The results are as shown in FIG. 7. The MDA-MB-231 cells in the cell control group can fill up the scratched region quickly through migration at 48 hours after scratching. Compared with the control group, healing of the scratched regions in different administration groups was inhibited and slowed down to different extents, especially in the (PTX+PSO+I-PSO)-PLN group, which was significantly inhibited, and the cells did not migrate significantly at 72 hours after scratching.

### Embodiment 7 Invasion Detection of MDA-MB-231 Cells Under Different Drug Components

(1) Preparation Before Experiment

After thawing Matrigel, 1 mL of Matrigel was taken out and placed in a refrigerator at 4° C. for standby service. A 200 μL nozzle, a serum-free DMEM culture fluid, a 24-well cell culture plate containing several Transwell and an operation box of an appropriate size were placed in a refrigerator at 20 degrees below zero for precooling overnight, so as to avoid the solidification of Matrigel due to the temperature rise during experimental operations to cause errors in the experiment. Another serum-free DMEM culture fluid containing 0.1% BSA was prepared for later use.

(2) Matrigel Coating

The entire experimental procedure was performed on ice to maintain a low temperature and to keep an eye on temperature changes anytime. An appropriate amount of Matrigel was taken out and diluted to a concentration of 300 ng/mL with the serum-free cell culture fluid. The diluted Matrigel was evenly injected into a Transwell chamber at 100 μl per well. When adding the Transwell, bubbles should be avoided and a device shall be kept horizontal. Then, the whole device was transferred to a cell incubator at 37° C. until the Matrigel was solidified, which took 30 minutes to 1 hour.

(3) Cell Preparation

Cell preparation could be started while waiting for the Matrigel to solidify. Adherent MDA-MB-231 cells were acted with different drug components including PTX, PSO, I-PSO, PTX+PSO+I-PSO and (PTX+PSO+I-PSO)-PLN in a DMEM-containing medium (10% fetal bovine serum) for 24 hours, digested with 0.25% pancreatin, and centrifuged at 1000 rmp/min for 2 minutes. A supernatant was discarded, and the remaining was washed with PBS for three times. The MDA-MB-231 cells were suspended in a 0.1% BSA serum-free DMEM medium, and the cells were counted with trypan blue and adjusted to a concentration of 1×10⁶ cells/mL.

(4) Cell Inoculation

The Transwell was taken out, 600 μL of complete medium containing 15% fetal bovine serum (tumor cell chemotactic factor) was added to a lower chamber firstly, then the Transwell coated with the Matrigel was carefully soaked in a well containing the culture fluid, then 100 μL (including 8×10⁴ cells) of each prepared cell suspension was taken and inoculated on the solidified Matrigel (avoiding bubbles), then the device was transferred to a 5% CO₂ cell culture fluid at 37° C. to incubate for 24 hours.

(5) Cell Staining

After incubation, the mixture was washed with PBS slightly to wipe off tumor cells and Matrigel that did not penetrate an upper surface of a filter membrane with cotton swabs. The Transwell was soaked in 1 ml of methanol and fixed for 10 minutes, and then stained with 500 μL of crystal violet for 5 minutes after air drying (staining with 0.1% crystal violet for 30 minutes after fixation). After the Transwell was washed with tap water (washed with PBS for three times), the cell distribution on a lower surface of the membrane was observed under a microscope.

(6) OD Value

500 μL of 33% acetic acid was added to the 24-well plate, and the chamber was placed in the plate, soaked in the membrane and shaken for 10 minutes for full dissolution, and then the chamber was taken out. An OD value at 570 nm of the 24-well plate was measured on an enzyme reader to indirectly show the cell number.

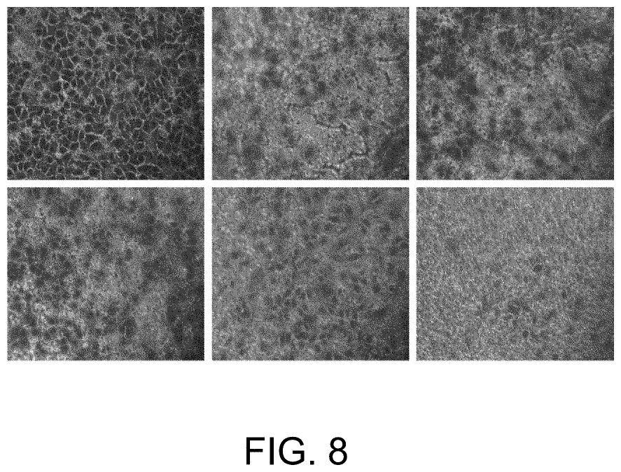

The results are as shown in FIG. 8. Compared with other drug administration groups, the cells of the (PTX+PSO+I-PSO)-PLN group penetrating the Transwell membrane were significantly reduced.

1. A composite nanoparticle, wherein the composite nanoparticle is a polymer-lipid nanoparticle encapsulating psoralen, isopsoralen and paclitaxel simultaneously.

2. The composite nanoparticle according to claim 1, wherein the composite nanoparticle has a particle diameter of 96.89±2.12 nm.

3. A preparation method of the composite nanoparticle according to claim 1, comprising the following steps of:
S1. dissolving soybean lecithin and DSPE-PEG2000 in an aqueous phase, subjected to blending and preheating; and
S2. dissolving psoralen, isopsoralen, paclitaxel and PLGA in an oleic phase, subjected to blending and injecting into the aqueous phase of the S1 to obtain a mixture, and then heating and blending the mixture to obtain the composite nanoparticle.

4. The preparation method according to claim 3, wherein the aqueous phase is absolute ethyl alcohol.

5. The preparation method according to claim 3, wherein the oleic phase is acetonitrile.

6. The preparation method according to claim 3, wherein a mass ratio of the soybean lecithin to the DSPE-PEG2000 in the S1 is M:1, and M is 5 to 6.

7. The preparation method according to claim 3, wherein a mass ratio of the psoralen to the isopsoralen to the paclitaxel in the S2 is 2:2:1.

8. The preparation method according to claim 3, wherein the heating and blending in the S2 refers to heating with stirring at 70° C. for 90 minutes.

9. A preparation method of anti-tumor drugs or composite nano preparations, comprising:
the use of the composite nanoparticle according to claim 1.

10. The preparation method according to claim 9, wherein the tumor is breast cancer.

11. A preparation method of the composite nanoparticle according to claim 2, comprising the following steps of:
S1. dissolving soybean lecithin and DSPE-PEG2000 in an aqueous phase, subjected to blending and preheating; and
S2. dissolving psoralen, isopsoralen, paclitaxel and PLGA in an oleic phase, subjected to blending and injecting into the aqueous phase of the S1 to obtain a mixture, and then heating and blending the mixture to obtain the composite nanoparticle.

12. The preparation method according to claim 11, wherein the aqueous phase is absolute ethyl alcohol.

13. The preparation method according to claim 11, wherein the oleic phase is acetonitrile.

14. The preparation method according to claim 11, wherein a mass ratio of the soybean lecithin to the DSPE-PEG2000 in the S1 is M:1, and M is 5 to 6.

15. The preparation method according to claim 11, wherein a mass ratio of the psoralen to the isopsoralen to the paclitaxel in the S2 is 2:2:1.

16. The preparation method according to claim 11, wherein the heating and blending in the S2 refers to heating with stirring at 70° C. for 90 minutes.

17. A preparation method of anti-tumor drugs or composite nano preparations, comprising:
the use of the composite nanoparticle according to claim 1.

18. The preparation method according to claim 17, wherein the tumor is breast cancer.
